(19) United States
(12) Patent Application Publication
(10) Pub. No.: US 2003/0114885 A1
(43) Pub. Date: Jun. 19, 2003

(54) System and device for implementing an integrated medical device component package

(76) Inventors: Richard C. Nova, Kirkland, WA (US); Shawn R. Bertagnole, Lake Stevens, WA (US); Douglas Brown, Kent, WA (US); Stephen L. Firman, Redmond, WA (US); William E. Saltzstein, Woodinville, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/262,536

(22) Filed: Sep. 30, 2002

Related U.S. Application Data
(60) Provisional application No. 60/331,358, filed on Oct. 2, 2001.

(51) Int. Cl. A61N 1/00

(52) U.S. Cl. 607/2

(57) ABSTRACT

A system and device for implementing an integrated component package in a medical device are provided. An integrated component package includes a set of components utilized by the medical device to deliver a therapy to patient and/or monitor a condition of a patient. The integrated component package can provide language-specific instructions, memory and power resources, and medical supplies for patient use. Further, the integrated component package can configure various medical device operational parameters on a medical device.

### CROSS-REFERENCE TO RELATED APPLICATION

This application claims the priority of U.S. Provisional Application No. 60/331,358, entitled SYSTEM AND DEVICE FOR IMPLEMENTING AN INTEGRATED MEDICAL DEVICE COMPONENT PACKAGE and filed on Oct. 2, 2001. U.S. Provisional Application No. 60/331,358 is incorporated by reference herein.

### FIELD OF THE INVENTION

This invention relates to medical devices, and in particular, to a system and device utilizing integrated component packages in defibrillator medical devices.

### BACKGROUND OF THE INVENTION

During a medical emergency, the ability to promptly diagnose and treat a patient with a medical device generally increases the likelihood of patient recovery. To decrease the amount of time between an emergency situation, such as a heart attack, and a first treatment, there is a growing trend to introduce medical devices to non-clinical settings, such as homes, businesses, schools, and other public places. Furthermore, some medical devices are designed to allow a minimally trained person to maintain the medical device and to administer emergency treatment to a patient. For example, in the case of portable defibrillators, such as a portable automated external defibrillator (“AED”), the defibrillator may be configured to be stored in non-clinical settings, such as a home or public building, and to allow a minimally trained person to attach a pair of electrodes to a patient's body and to initiate the delivery of a therapeutic shock in emergency situations. Because the portable defibrillator is targeted to be used by a minimally trained person, various aspects of the portable defibrillators are designed to be relatively inexpensive, easy to use and maintain, and user friendly.

In one aspect for facilitating the use of a portable defibrillator by a minimally trained person, the portable defibrillator provides voice and textual prompts to a minimally trained individual instructing the individual how to apply the therapy device to the patient's body, how to perform basic cardiopulmonary resuscitation (“CPR”) and how to operate the portable defibrillator to deliver the therapeutic shock if required. The ability of the defibrillator to quickly and clearly convey the use instructions to the untrained individual is essential to ensure the device is utilized properly. Thus, the language of the defibrillator instructions becomes important to the effectiveness of defibrillator operation.

In one conventional embodiment, a defibrillator may be configured to provide instructions in a single language, such as English. One skilled in the relevant art will appreciate that the effective area of distribution of the single language defibrillator, such as English only, is limited to regions in which the English is the primary language. Additionally, even in regions where English is a primary language, the effectiveness of the defibrillator would be diminished if a particular individual could not understand the selected language instructions. Thus, to distribute a defibrillator to different regions, a defibrillator manufacturer would have to provide and maintain different language versions of the same defibrillator model. This approach increases the overall cost of developing and maintaining multiple language versions of the same defibrillator model, and inhibits the wide distribution of the defibrillator model to non-traditional locations, such as homes, schools and businesses.

One approach to providing a defibrillator model with a wider distribution entails equipping each defibrillator with multiple language modules. Each language module contains language-specific resources that allow a minimally trained person to select a preferred language to receive instructions/display interfaces. While the multiple language module approach allows a single defibrillator model to be more widely distributed, the approach remains deficient in that each defibrillator would be required to have higher memory resources to store and maintain multiple language modules, even if they are not being utilized. Additionally, the defibrillator would also require some type of user interface to allow for the selection of a particular language module. Moreover, because the defibrillator includes a large set of language modules, updates to the software modules are more burdensome and increase the cost of maintaining the defibrillator. Accordingly, the additional memory resources and maintenance cost increases the overall cost of each defibrillator and decreases the ease of maintaining the defibrillator software code.

In another aspect for facilitating the use of a portable defibrillator by a minimally trained person, a defibrillator typically includes a mechanism for updating the existing software code base. For example, one skilled in the relevant art will appreciate that it may be necessary to update, supplement, or modify the underlying software code base within a portable defibrillator. In one typical embodiment, updating the software code base requires the replacement of a defibrillator memory storage unit containing the previous software code with a new memory storage unit containing the updated software code base. This approach is deficient in that the defibrillator must be taken to a maintenance center or alternatively, a service technician must travel to often remote locations solely to replace the memory storage unit.

In another typical embodiment addressing defibrillator software updates, the defibrillator may include a rewriteable memory storage unit that allows a remote monitoring service to transfer software update data via a robust communication system, such as a wired or wireless communication network. While this embodiment mitigates the need for replacing a memory storage unit for each software update, an adequate communication system utilized to update the software may not available in all regions. For example, if a portable defibrillator were located in a building that receives poor wireless network reception, the defibrillator would be unable to receive the software update. Moreover, the overall cost of the defibrillator is increased by the additional components and software enabling the defibrillator to establish communication with the remote monitoring service.

In a further aspect for facilitating the use of a portable defibrillator by a minimally trained person, the defibrillator can include additional memory components that allow retrieval and storage of patient data. For example, comparing the results of a diagnostic test with previous patient data may be extremely useful in diagnosing whether an additional therapy treatment is necessary. In accordance with one embodiment for storing patient data, the defibrillator is connected to an external data storage source, such as a tape drive or a Personal Computer Memory Card International Association (“PMCIA”) storage card. Alternatively, the defibrillator may store the patient data internally and transmit the data to a remote storage location via a communication channel. Although such external memory components embodiments can be utilized to store patient data, the use of typical external storage memory components in portable defibrillators remains deficient in that the external data storage is a separate, independent function performed by the defibrillator. Accordingly, the external memory component generally increases the complexity and cost of the portable defibrillator.

In yet another aspect for facilitating the use of a portable defibrillator by a minimally trained person, the defibrillator power source must be sufficiently reliable to energize the various defibrillator components and to provide a sufficient amount of energy to deliver one or more therapeutic shocks. One skilled in the art will appreciate that there are numerous power source monitoring components and algorithms to monitor the state of a defibrillator power source. As applied to a low cost, portable defibrillator, the conventional battery monitoring components and algorithms become deficient because they can increase the overall cost of each defibrillator in terms of development and maintenance. Additionally, in the event the monitoring components and algorithms detect an insufficient power source, a minimally trained person must first recognize that the defibrillator power source has become inoperative and either attempt to replace the power source or request a trained technician to replace the power source.

In still a further aspect for facilitating the use of a portable defibrillator by a minimally trained person, a portable defibrillator may need to be configured to allow for multiple uses before the defibrillator can be inspected and reconditioned. One skilled in the relevant art will appreciate that after a therapy session, it may undesirable, or impossible, to reuse some of the defibrillator components that have direct contact with the patient. For example, a portable defibrillator typically utilizes a set of disposable electrodes to deliver the therapeutic shock. Utilizing the same set of disposable electrodes on different patients without proper sterilization may present a health risk. Additionally, a set of disposable electrodes are either designed to or otherwise may become inoperative after a single use for a variety of reasons, especially if the defibrillator is not located in a clinical setting. Although replacement defibrillator components may be available, this approach becomes deficient by requiring the minimally person to locate replacement parts and properly replace the worn or used components.

Although each individual aspect for facilitating the use of a portable defibrillator by a minimally trained person can be addressed, at least in part by some conventional embodiments, the grouping of some or all the multiple aspects also presents a deficiency not properly addressed with the prior art. For example, providing a defibrillator that has multiple language modules, power source monitoring components, and external data storage amplifies the cost deficiencies associated with each aspect for facilitating use by the minimally trained person.

Thus, based on the above-described deficiencies associated with the conventional art, there is a need for an integrated component package that can provide any one of a combination of functions facilitating use of a portable defibrillator.

### SUMMARY OF THE INVENTION

A system and device for implementing an integrated component package in a medical device are provided. An integrated component package includes a set of components utilized by the medical device to deliver a therapy to patient and/or monitor a patient condition. The package can provide language-specific instructions, memory and power resources, and medical supplies for patient use. Further, the integrated component package can configure various medical device operational parameters.

In accordance with aspects of the present invention, an integrated component package is provided. The integrated component package includes one or more medical device components operable to be utilized in conjunction with the operation of a medical device, and at least one transducer device operable to communicate with the medical device.

In accordance with another aspect of the present invention, a medical device is provided. The medical device includes an external medical device component interface operable to a medical device component associated with an integrated component package and an external transducer interface operable to be connected to an external transducer device associated with the integrated component package. The external medical device component interface and the external transducer interface are operable to interface with one or more external component packages.

In accordance with a further aspect of the present invention, a medical device system is provided including a medical device having an external medical device component interface and an external transducer interface. The medical device system also includes an integrated component package having one or more medical device components operable to be utilized in conjunction with the operation of the medical device, and a transducer device operable to communicate with the external transducer interface of the medical device. The integrated component package is removably coupled to the medical device.

In accordance with still a further aspect of the present invention, a medical device system is provided. The medical device system includes a medical device having power interface means and transducer interface means. The medical device system also includes an integrated component package having power source means to deliver a power source to the medical device and transducer means to communicate with the transducer interface means. The medical device and the integrated component package are removably coupled.

In accordance with yet another aspect of the present invention, an integrated component package is provided. The integrated component package includes one or more medical device components operable to be utilized in conjunction with the operation of a medical device, and at least one transducer device operable to communicate with the medical device. The integrated component package further includes a programmable data source having language-specific data stored thereon. The language-specific data is operable to generate a language-specific user, interface on the medical device. The integrated component package also includes a data interface operable to transmit the language-specific data to the medical device.

In accordance with still a further aspect of the present invention, a medical device is provided. The medical device includes an external transducer interface operable to communicate with a transducer set, and an external data interface operable to obtain language-specific data from a programmable data source. The the external transducer interface and the external data interface are operable to interface with one or more external component packages.

In accordance with still another aspect of the present invention, a medical device system is provided including a medical device having an external medical device component interface, an external transducer interface, and an external data interface. The electrotherapy system also includes an integrated component package having one or more medical device components operable to be utilized in conjunction with the operation of the medical device, a transducer device operable to communicate with the external transducer interface of the medical device, and a programmable data source having language-specific data operable to generate a language-specific interface on the medical device. The programmable data source transmits the language-specific data to the external data interface of the medical device. The integrated component package is removably coupled to the medical device.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a pictorial view illustrating a top perspective of an integrated component package formed in accordance with the present invention;

FIG. 2 is a pictorial view illustrating a bottom perspective of an integrated component package formed in accordance with the present invention;

FIG. 3 is an exploded perspective view of an integrated component package containing various components formed in accordance with the present invention;

FIG. 4 is a block diagram of a defibrillator for use with an integrated component package formed in accordance with the present invention; and

FIGS. 5-8 are pictorial views illustrative of an interface between a defibrillator medical device and an integrated component package formed in accordance with the present invention.

### DETAILED DESCRIPTION OF THE PREFERRED EMBODIMENT

The present invention is directed to a system and device for implementing an integrated component package for use with medical devices. Specifically, the present invention will be described in relation to a system and device for utilizing an integrated component package in a portable automated external defibrillator (“AED”) to provide the AED with one or more components used during the operation of the AED. As will be readily understood by one skilled in the relevant art, the present invention is not limited in its application to an integrated component package for an AED. For example, the present invention may be applied to manual defibrillators, and other kinds of medical devices such as monitoring devices (e.g., electrocardiogram (ECG) monitors, blood pressure monitors, SpO₂ monitors, pulse monitors, electroencephalogram (EEG) monitors, end tidal CO₂ monitors, etc.) drug infusion devices, catheter-based devices, and other therapy devices (e.g., electrosurgery devices, cardiac pacing, cardiopulmonary resuscitation (CPR) assistance devices, and the like). Thus, it is to be understood that the disclosed embodiments are presented by way of example and should not be construed as limiting.

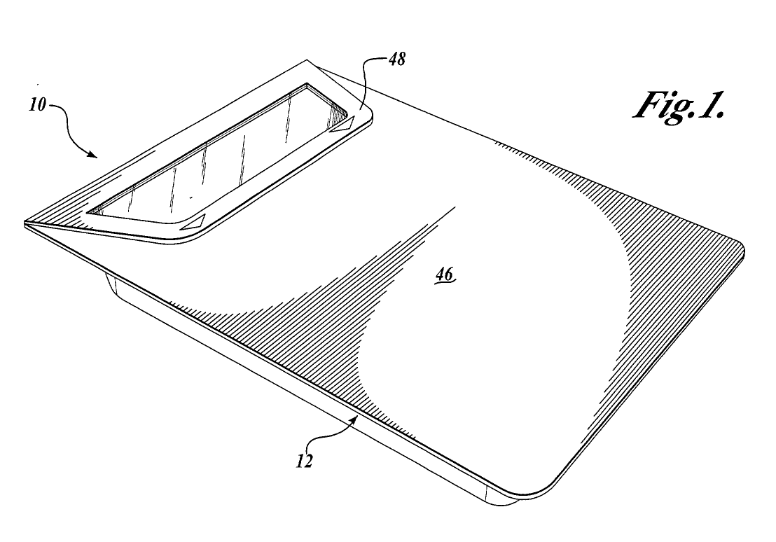

FIGS. 1-3 are pictorial views illustrative of an integrated component package 10 for use with an AED in accordance with an embodiment of the present invention. As illustrated in FIGS. 1-3, the integrated component package 10 includes an outer shell 12 that defines a generally rectangular shape. In one embodiment of the present invention, the outer shell 12 is constructed of a plastic type material sufficient to substantially keep the shape of the integrated component package 10. The outer shell 12 includes four side walls 14, a bottom surface 16, and a lip 18 that define a storage bay 20. Those of ordinary skill in the art will appreciate that the outer shell 12 may be of any shape, dimension or configuration and may be made of any material suitable for use with any desired portable medical device without departing from the spirit and scope of the present invention.

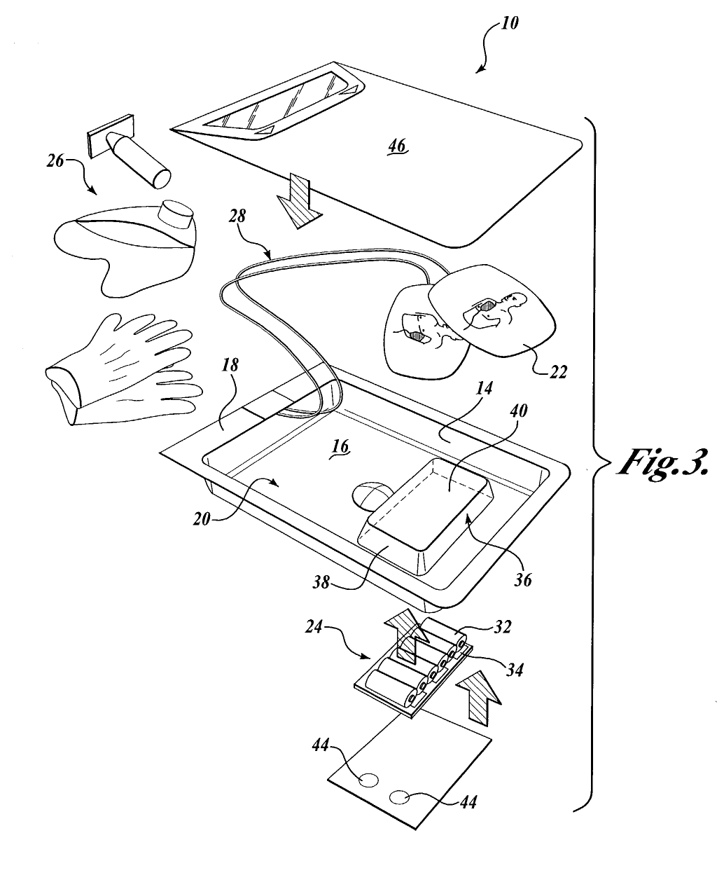

As illustrated in FIG. 3, for a medical device such as a portable AED, the storage bay 20 may house a pair of transducers 22, a combination battery pack and memory chip set 24 and assorted medical supplies 26, such as a CPR mouth to mouth barrier, a safety razor, and protective gloves, etc. The storage bay 20 may also include additional medical device components not illustrated in FIG. 3, including, but not limited to language specific text instructions, scissors, alcohol swabs, oral drugs/medications, injectable drugs/medications, transdermal drugs/medications, towels, light sources (e.g., flashlight), writing utensils, earphones, eye protectors, timing devices (e.g., stopwatch), microphones, input devices (e.g., keypads), recording devices, patient data storage devices, one or more additional pairs of transducers and/or communications devices (e.g., mobile telephones or radios).

In an illustrative embodiment of the present invention, the pair of transducers 22 are single-use disposable electrodes that are operable to receive a therapeutic shock signal from an AED and deliver the therapeutic shock signal to a patient. Additionally, the pair of transducers 22 may be further operable to obtain patient information, such as an ECG signal, blood pressure, pulse, etc. for use by the AED. Each electrode in the pair of transducers 22 is connected to the AED via a set of conductive wires 28. In an illustrative embodiment of the present invention, the conductive wires 28 are connected to an electrical therapy interface 30 that is located within one of the side walls 14 of the outer shell 12. The relationship of the electrical therapy interface 30 with an AED will be explained in greater detail below. Thus, the pair of transducers 22 are physically connected to the integrated component package 10 and the conductive wires 28 are of a sufficient length to allow placement of the pair of transducers 22 on the body of a patient. One skilled in the relevant art will appreciate that multiple use electrodes may also be utilized within the integrated component package 10. Moreover, the pair of transducers 22 may be detachable from the integrated component package 10 to remain with the patient. Still further, the integrated component package 10 may include additional pairs of transducers to separate the monitoring and therapeutic delivery functions or add additional functions, such as transducers sized for pediatric or infant use.

In an actual embodiment of the present invention, the combination battery pack and memory chipset 24 may include a power source 32 aspect that utilizes one or more batteries for supplying energy to an AED. The power source 32 can include a single use battery (or set of batteries) or a multiple use rechargeable battery (or set of batteries).

The combination battery pack and memory chipset 24 may also include a memory/software chipset 34 aspect operable to store computer-readable instructions or data utilized by an AED's on-board processor. The computer-readable instructions and data can be referred to generally as medical device data. The memory/software chipset 34 may comprise electronically erasable read-only memory (“EEROM”), FLASH read-only memory (“ROM”), dynamic random access memory (“DRAM”), static random access memory (“SRAM”), other varieties of volatile and nonvolatile storage components, or any combination thereof. Additionally, the memory/software chipset 34 may utilize the power source 32 to preserve the contents stored in volatile memory components.

In accordance with one embodiment of the present invention, the memory/software chipset 34 stores computer-readable instructions and/or data that are embodied as a set of language-specific modules specifically configured to allow an AED to provide user instructions and displays in a predetermined language. One skilled in the relevant art will appreciate that a processor within an AED, or otherwise connected to the AED, utilizes computer-readable instructions and/or data to generate language-specific instructions to a user, such as through a loudspeaker or on a visual display. For example, an AED processor may be programmed to display an introduction screen upon activation of the AED by recalling a software module containing the introduction screen from memory. In accordance with a language-specific embodiment, the AED would retrieve a selected language-specific module from the memory/software chipset 34 to generate the appropriate instructions and displays. The AED may load some or all of the language-specific data modules into an internal AED memory for later use or, alternatively, the AED may load a specific module as it is needed from the memory/software chipset 34.

In an alternative embodiment of the present invention, the memory/software chipset 34 may store multiple sets of language-specific modules to allow an AED to provide two or more language interfaces. For example, the memory/software chipset 34 may include a grouping of languages common to a particular region. Accordingly, a user can select one of the languages by manipulating an interface on the AED or an interface provided in the integrated component package 10.

In another alternative embodiment, the AED may store one or more language-specific computer-readable modules in an internal memory. Accordingly, the AED does not load language-specific modules from the memory/software chipset 32 to provide a language-specific interface. Instead, the memory/software chipset 32 may include computer-readable data that specifies which of the set of language-specific modules stored within the AED should be utilized to provide audio and video displays and instructions.

Thus, in accordance with the above-disclosed embodiments, a user may select a language for the AED display and audio instructions by selecting the appropriate integrated component package 10 that can be identified to provide a particular language or subset of languages, by printing the available selected language(s) on the outside of the integrated component package 10 or by some other labeling method.

In another embodiment of the present invention the memory/software chipset 34 may include computer-readable instructions that update or replace previously installed computer-readable instructions within the AED. One skilled in the relevant art will appreciate art that an AED processor utilizes computer-readable instructions for general operating functions and to communicate with various hardware components that are described in greater detail below. These computer-readable instructions are commonly referred to as an operating environment or the underlying software. In accordance with this embodiment, the memory/software chipset 34 includes computer-readable instructions and/or data that replaces a portion or all of the operating environment or underlying software previously stored within the AED. For example, the integrated component package 10 may include an operating environment update that modifies an algorithm that is utilized to diagnose a patient, or that includes additional computer-readable instructions to allow the AED to communicate with a new hardware component. In one illustrative example, upon interfacing with an integrated component package 10, the AED would examine the memory/software chipset 34 and would automatically download and implement any operating environment updates found stored in the chipset 34. Alternatively, the user may manipulate the AED, such as through an interface, to cause it to search and/or download the operating environment updates.

By including the computer-readable instructions that update the operating environment or underlying software with the integrated component package 10, a manufacturer can update AED computer-readable instructions without requiring a robust communication network or memory chip replacement. Moreover, in some embodiments, the computer-readable instructions may be processed without requiring additional user participation.

In accordance with yet another embodiment, the memory/software chipset 34 includes computer-readable instructions that are operable to modify one or more operational parameters within an AED. One skilled in the relevant art will appreciate that, in addition to the computer-executable instructions utilized to control the general operating and communication functions described above, an AED may also receive computer-executable instructions that vary various operational parameters of an AED. The operational parameters do not modify the underlying processes utilized by an AED, but rather select specific values for parameters provided by the AED. For example, AED operational parameters can include a selection of a specific range of energy levels for a therapeutic shock signal or minimum delay between the delivery of therapeutic shock treatments. Other operational parameters include expected ranges of patient diagnostic indicators that are utilized by diagnostic algorithms stored in the AED to determine whether therapeutic shock therapy is appropriate. The patient diagnostic indicators can include an expected pulse rate, expected blood pressure range, body temperature, impedance, etc.

In an illustrative example, the memory/software chipset 34 of the integrated component package 10 may include various computer-executable instructions that adjust various AED operational parameters according an approximate weight, size, or age of a patient when loaded by the AED. Accordingly, by including the parameters for which each integrated component package 10 is optimized for, a minimally trained person can assist a patient's treatment by selecting an integrated component package 10 that corresponds generally with an observed range of weight, size, or age, such as for an adult or a child.

In a still further embodiment of the present invention, the memory/software chipset 34 may also include additional readable/writable memory components for transmitting and receiving patient data. Patient data may include patient diagnostic data such as ECG data, a detected pulse rate, or blood pressure. Patient data may also include treatment data such as a number of therapeutic shock signals delivered, the energy levels for each therapeutic shock signal delivered, the time and date of delivered therapies, and any resulting diagnostic indicators. Patient data may also include any additional data uploaded by a user, such as general observations, through an input device, including keyboards, microphones, or other computing devices. The patient data may be downloaded to another device by a treating physician, or other medical personnel, to provide a more detailed description of the patient's medical history. For example, a physician may download the information from the memory/software chipset 34 with a personal computing device to review the patient data. The patient data may also be utilized to determine whether previous therapeutic shock signal energy levels were effective.

In one embodiment of the present invention, the memory/software chipset 34 may be utilized for a variety of storage purposes (such as software update, configuration changes and patient data). Alternatively, the patient data may be stored in a separate portion of the memory/software chipset 34, or as a separate component. Moreover, the integrated component package 10 may be configured to facilitate the removal of the patient data from a spent integrated package 10. The configurations can include additional output ports for connecting to another device or physical devices to allow for the removal of all or a portion of the memory/software chipset 34. Further, the configurations can include wireless transmission components for transmitting the patient data. The memory/software chipset 34 may have stored on it patient data taken from one or more previous uses. Accordingly, the memory/software chipset 34 may overwrite a portion or all previously stored patient data, or it may add to a previously stored data.

In accordance with the present invention, the selection of the function of the memory/software chipset 34 may be set on the function of the integrated component package 10. Alternatively the function of the memory/software chipset 34 may be set by a user.

In an actual embodiment of the present invention, the outer shell 12 of the integrated component package 10 defines a housing 36 for holding the combination battery pack and memory chipset 24. As illustrated in FIG. 3, housing 36 includes four side walls 38 and a top surface 40 such that the bottom surface 16 of the outer shell 12 provides an opening for accepting the combination battery pack and memory chipset 24. The housing 36 provides physical protection to the combination battery pack and memory chipset 24 from other components in integrated package 10. The housing 40 also serves to provide at least some form of electrical isolation for the components of the combination battery pack and memory chipset 24. One skilled in the relevant art will appreciate that the housing 36 may also be lined with additional shielding materials. In an alternative embodiment of the present invention, in the event the combination battery pack and memory chipset is divided into individual components 32, 34, the housing 36 may also provide separate compartments for the components 32, 34.

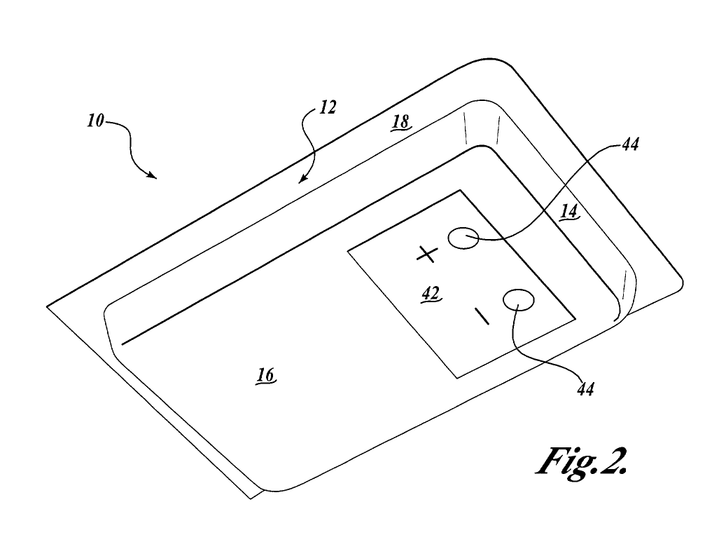

With reference to FIGS. 2 and 3, the combination battery pack and memory chipset 24 are contained within the housing 36 by a cover 42. In one embodiment of the present invention, the cover 42 seals the opening created by the housing 36 to secure the combination battery pack and memory chipset 24 within the outer shell 12. The cover 42 further includes a set of electrical contacts 44 for establishing a power/data interface between the combination battery pack and memory chipset 24 and an AED. One skilled in the relevant art will appreciate that any one of a variety of electrical contacts may be utilized to transmit the power and/or data signals between the combination battery pack and memory chipset 24 and an AED. Moreover, the cover 42 may also include a separate contact or set of contacts for transferring data and for transferring power, in which each contact is optimally configured for a particular function (data delivery, power delivery). For example, the contacts 44 may include plated electrical contacts, optical contacts, magnetic contacts, or a combination thereof. The power/data interface with an AED will be explained in greater detail below.

In accordance with another embodiment of the present invention, a portion of the bottom surface 16 of the outer shell 12 may be transparent or translucent to provide a window for viewing display data from the AED. Moreover, in another embodiment, the bottom surface 16 may include a visual generating device, such as a liquid crystal display (“LCD”) to generate display data. Still further, the bottom surface 16 of the outer shell 12 may also have raised areas, holes, or other objects to facilitate the pressing of any AED interfaces, such as buttons, select switches and the like. A detailed description of these interfaces will be described below.

The integrated component package 10 also includes a releasable liner 46 operable to seal selected components within the storage bay 20. The releasable liner 46 can include a handle 48 to aid in removing the releasable liner 46 from the outer shell 12. The releasable liner 46 may be removably attached to the lip 18 of the outer shell 12 for preserving the sterility of the components, such as the medical supplies 26 or the pair of transducers 22.

In the embodiment illustrated in FIGS. 1-3, the integrated component package 10 is configured to integrate with an AED in a top down fashion as will be explained in greater detail below. One skilled in the relevant art will appreciate that the integrated component package 10 may also be configured to integrate with an AED in a variety of other manners. For example, the integrated component package 10 may be in the form of a cartridge or plug-in component that is inserted into an opening within an AED such that it is either fully or partially housed within an inner portion of the AED. The integrated component package 10 may also be in the form of a module that is separate from the AED and establishes one or more external data and power connections via conductive wires without integration with the AED. All are considered to be within the scope of the present invention.

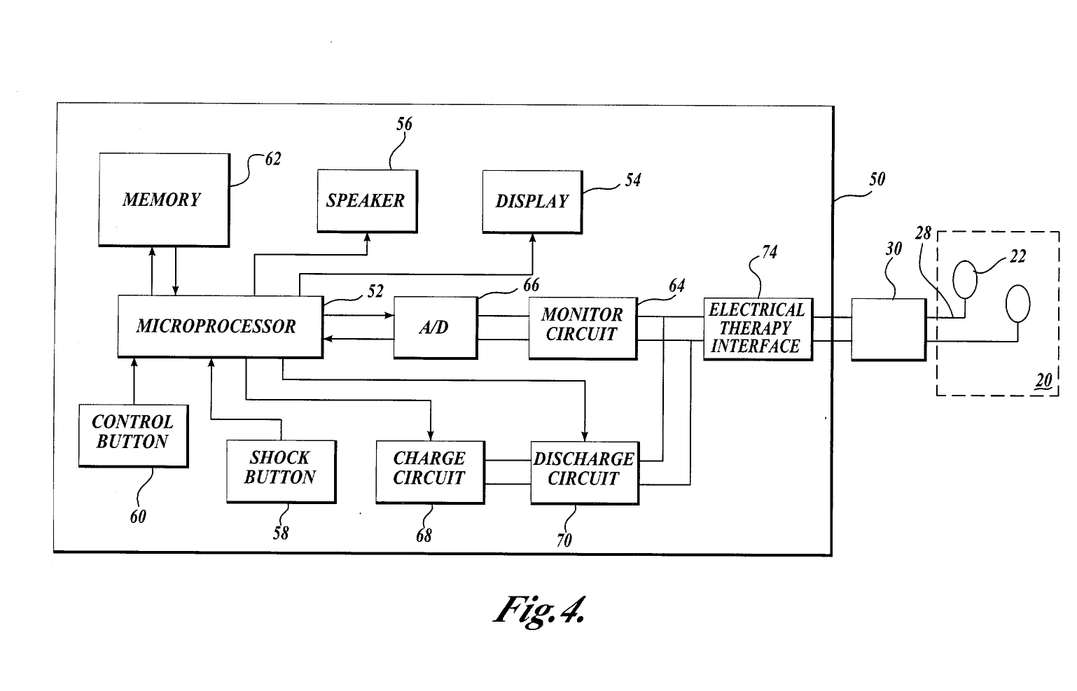

FIG. 4 is a block diagram illustrative of a portable automated defibrillator 50 for use with an integrated component package 10 in accordance with the present invention. Although a defibrillator is illustrated in this embodiment, those skilled in the art will be able to implement other embodiments using other types of medical equipment, without undue experimentation. Moreover, the integrated component package 10 may be integrated with other variations of defibrillator medical devices.

As shown in more detail in FIG. 4, the AED 50 includes a microprocessor 52 that controls the operation of the AED 50. The microprocessor 52 is connected to a display 34, a speaker 56, a shock button 58, and a control button 60. The microprocessor 52 is also connected to a memory 62 that stores computer-executable operating instructions, computer-executable operational parameters, and other computer-executable instructions in accordance with the present invention. For example, the memory 62 may also store language modules for generating language-specific user interfaces. The memory 62 may also store patient use data for transfer to the integrated component package 10, or other devices.

In accordance with an illustrative embodiment of the present invention, during defibrillation operation, the microprocessor 52 analyzes an electrocardiogram (ECG) of a patient using an automatic heart rhythm detection algorithm also stored in memory 62 to identify whether the patient is experiencing a shockable heart rhythm, such as ventricular fibrillation. The detection algorithm executed by the microprocessor 52 in the actual embodiment of the present invention described herein is similar to that used in the LIFEPAK® 500 defibrillator provided by Medtronic Physio-Control Corp. of Redmond, Wash. Other known heart rhythm detection algorithms may also be used without departing from the scope of the present invention, such as those algorithms designed to comply with standards promulgated by the Association for the Advancement of Medical Instruments (AAMI). The ECG signals analyzed by the detection algorithm are collected by the electrode pair 22 and passed through a monitor circuit 64 via the electrical therapy interface 32 to an analog-to-digital converter 66. The analog-to-digital converter 66 then passes the digitized signals to microprocessor 52. If the microprocessor 52 detects a shockable rhythm, the microprocessor causes a charging circuit 68 to generate a current causing a storage capacitor (not shown) to charge in preparation for delivery of a defibrillation pulse. When the capacitor is fully charged, and delivery of the defibrillation pulse initiated, a discharge circuit 70 coupled to the microprocessor 50 and charge circuit 68 discharges the defibrillation pulse to the transducers 22 via the electrical therapy interface for application of the defibrillation pulse to the patient.

In one embodiment of the present invention, the charge circuit 68 is implemented with a capacitor with a capacitance of about 190-200 μF. The discharge circuit 70 is implemented in an H-bridge configuration, which facilitates generating biphasic output pulses. For example, the discharge circuit 70 can be implemented as disclosed in commonly-owned U.S. Pat. No. 5,824,017, entitled “H-Bridge Circuit For Generating A High-Energy Biphasic Waveform In An External Defibrillator” to J. L. Sullivan et al.

As described above, in one embodiment of the present invention, the integrated component package 10 may be utilized to provide the AED 50 with one or more components that are typically required for the proper function of the AED. For example, the AED 50 may be required to obtain power solely from the combination battery pack and memory chipset 24 of the integrated component package 10. Additionally, the AED 50 may only be able to deliver a therapeutic shock signal through the electrode pair 22 of the integrated component package 10. In another embodiment of the present invention, the AED 50 may have one or more components that are supplemented by components provided by the integrated component package 10. In accordance with this embodiment, the AED 50 may also include a power source component, a pair of transducers, or language modules in memory 62 that may be utilized. However, if an integrated component package 10 includes components that are redundant to any components on the AED 50, the AED 50 by default may utilize the integrated component package 10 components, or upon receiving some instruction from a user.

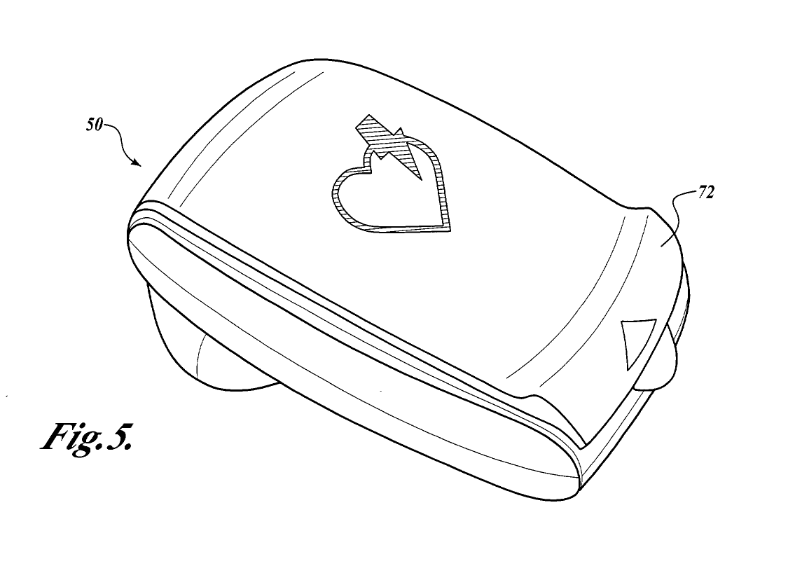

FIGS. 5-8 illustrate an AED suitable for use with an integrated component package 10 formed in accordance with an illustrative embodiment of the present invention. One skilled in the relevant art will appreciate that the interface between an integrated component package 10 and the AED 50 will vary depending on the configuration of the integrated component package 10, e.g., top-down cartridge, plug-in module, etc., and the components contained within the integrated package 10. Accordingly, the disclosed embodiment is illustrative and should not be construed as limiting.

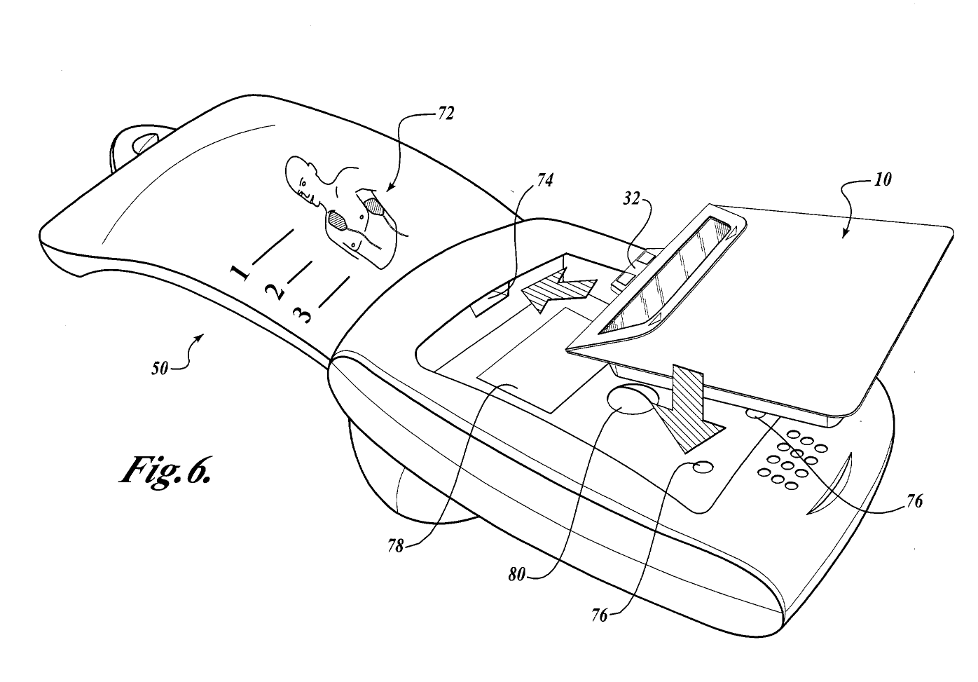

Referring now to FIG. 5, the AED 50 may be configured with protective components, such as a lid 72, during storage. In an actual embodiment of the present invention, the lid 72 of the AED 50 protects various interface components utilized by an integrated component package 10 such as electrical and data interfaces, display screens, and input devices (shock button). The lid 72 may be fixedly removable, or, as illustrated in FIG. 6, the lid 72 may be hinged to provide for opening. One skilled in the relevant art will appreciate that the size and function of the lid 72 depends on the configuration of the integrated package. In alternative embodiments of the present invention, a user may have to take other actions, such as depressing buttons or sliding panels, to access AED interface components.

At the same time the AED 50 is prepared by removing any protective coverings, the user selects an appropriate integrated component package 10 appropriate for the AED's intended use. Depending on the components contained within the integrated component package 10, the user selects an appropriate integrated component package based on a variety of factors including but not limited to language, physical aspects of the patient, data capabilities, model of AED, and the like. Some integrated component packages 10 may be configured to be used in situations meeting specific criteria such as integrated component packages personalized for particular individuals, desired language, or other criteria, while other integrated component packages may be configured for more generic use. Additionally, the integrated component package 10 may include color coding, language-specific text and graphs to assist a minimally trained person in selecting an appropriate integrated component package 10.

With reference to FIG. 6, once the lid 72 is removed or opened, the user is presented various interfaces and displays that are utilized to connect the AED 50 to the integrated component package 10 and to facilitate use of the AED 50 by the user. The AED 50 can include an electrical therapy interface 74 that corresponds to the electrical therapy interface 32 of the integrated component package 10. As illustrated in FIG. 6, the electrical therapy interface 32 of the integrated component package 10 has an outwardly extending portion having at least some conductive material to make an electrical contact with the electrical therapy interface 74 of the AED 50. The interfaces 32, 74 may also include threads, locking mechanisms, or other conductive interfacing components. One skilled in the relevant art will appreciate that any one of a variety of electrical interface components may be utilized by the present invention to electrically couple the integrated package 10 to the AED 50.

Another interface provided by the AED 50 includes one or more electrical contacts 76 for interfacing with the electrical contacts 44 connected to combination battery pack and memory chipset 24 of the integrated component package 10. In accordance with the present invention, the electrical contacts 76 facilitate the transfer of power from the power source 32 and/or the transfer of data to/from the memory/software chipset 34. Accordingly, the contacts 76 may be configured to facilitate either of these functions, such as to include optical transmission components or having plated external contacts. Moreover, the electrical contacts 76 of the AED 50 or the electrical contacts of the integrated component package 10 may include externally protruding components to facilitate the integration of the integrated component package 10 with the AED 50.

Other interfaces provided by the AED 50 includes a display screen 78 for generating graphical and textual messages to assist in using the AED 50. Accordingly, the integrated component package 10 would include at least some translucent or transparent portion of the bottom surface 16 of the shell 12 that would allow a user to view the display screen 78. Additionally, the AED 50 may also provide one or more external input devices, such as a shock button 80, that allow a user to input signals to the AED 50. The integrated component package 10 may include cut-out portions or raised portions that allow the use of those types of input devices. One skilled in the relevant art will appreciate that the display screen 78 or shock button 80 may also be positioned so as to not interfere with the utilization integrated component package 10.

In an illustrative embodiment of the present invention, the lid 72 of the AED 50 may include text and graphics that assist a minimally trained user in aligning the integrated component package 10 with the AED. Additionally, the integrated component package 10 may also have additional text and graphics (not shown) that assist a user in aligning the interfacing components of the integrated component package 10 with the AED 50, including but not limited to, color coded objects, arrows, and the like. Still further, some interface components, such as the therapy interface 74, may be interfaced in a specific order to assist the aligning of additional components.

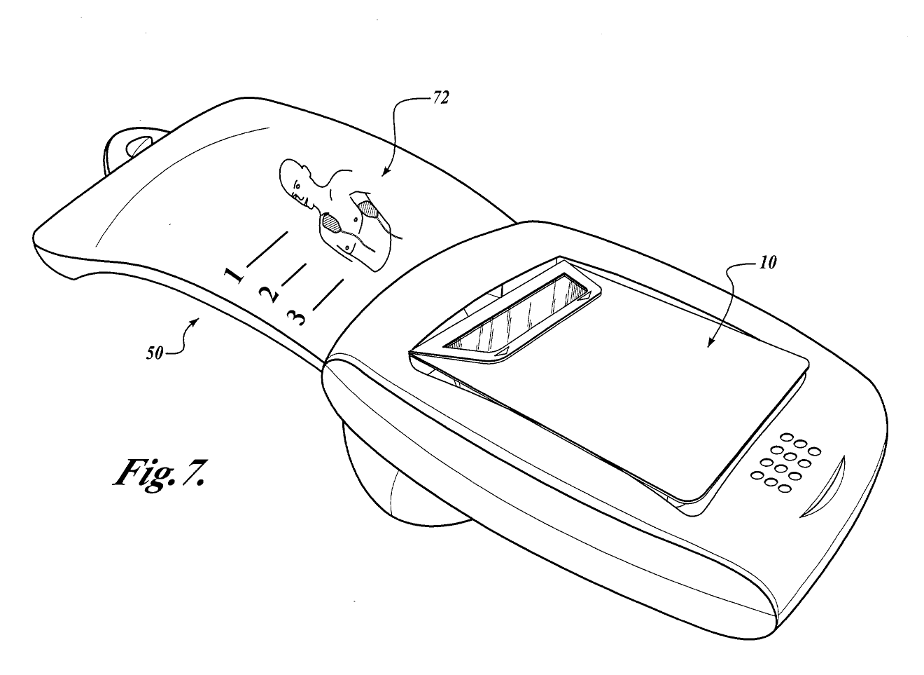

Referring now to FIG. 7, once the various interfaces are joined, the integrated component package 10 fits flush against a top surface of the AED. The integrated component package 10 and the AED 50 may include clips or other fasteners that hold the integrated component package to the AED and that make an audible sound when they are properly or not properly connected. Moreover, the AED 50 may also include one or more visual indicators, such as a light emitting diode (“LED”), that signals when a proper interface with an integrated component package 10 has occurred and/or failed.

In an illustrative embodiment of the present invention, the AED 50 microprocessor 52 may be configured to recognize when an integrated component package 10 is connected to an AED and to conduct various interface tasks upon a successful connection. For example, the microprocessor 52 may conduct a test of the AED components and the included integrated component package components to ensure that they are working and interface properly. The microprocessor 52 may also instigate the transfer of computer-readable instructions, operational parameter selections, and patient use data to/from the AED memory 62. Accordingly, the microprocessor 52 may read from the memory/software chipset 34 or it may directly download the data to internal AED memory 62 for processing. Alternatively, the microprocessor 52 may issue a prompt to the user to activate one or more of these functions. In an additional embodiment of the present invention, the integrated component package 10 may include serial number information, date of creation or other identification information, that can inform the microprocessor whether the integrated component package 10 is compatible with the AED 50 or whether the integrated component package 10 has an expiration date that has passed. Still further, the AED 50 may be configured to perform a self-test of all components upon the insertion of an integrated component package.

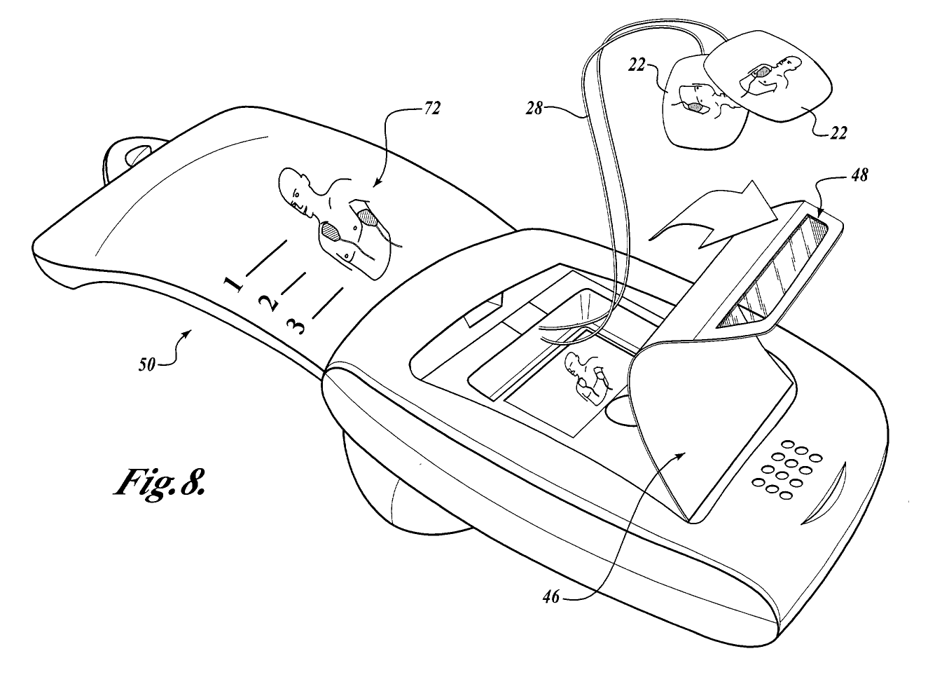

Referring now to FIG. 8, once the integrated component package 10 is secured to the AED 50, the components within the integrated component package 10 may be accessed by removing the releasable liner 46. As illustrated in FIG. 8, a user may pull back the releasable liner 46 by lifting the handle 48. Additionally, in one embodiment of the present invention, the AED may be activated upon the removal of the releasable liner 46, the lid 60, or upon a user input such as an on/off button. Upon activation, the AED 50 will function according to the software code within the AED or perhaps according to software stored on the memory/software chipset 34.

In one embodiment of the present invention, an integrated component package 10 may be limited to a single use to prevent multiple use of the components. For example, the integrated component package may include breakable clips that prevent subsequent interfaces with the same or a different AED 50. Additionally, the AED 50 microprocessor 52 may include additional computer-readable instructions that cause all or a portion of the data stored in the memory/software chipset 34 to be erased or blocked after the AED 50 has utilized the data.

In an alternative embodiment of the present invention, the integrated component package 10 may be configured to follow a particular patient through various aspects and locations of treatment. In accordance with this embodiment, an integrated component package 10, potentially including patient use data gathered and inputted by the AED 50 or the patient and stored within the memory/software chipset 34, follows the patient through various stages of treatments. For example, a patient may be given a first treatment in a public place from a portable AED 50, where a first set of data is acquired. The integrated component package 10 may be removed from the portable AED and transferred to an emergency response team AED, a defibrillator, medical device, or other computing system operable to interface with the integrated component package 10, where a second set of patient use data is acquired. The integrated component package 10 may be removed and transferred to another destination, where the first two sets of patient use data may be utilized to diagnose additional therapy or to track patient progress. In the above example, another defibrillator or medical device may utilize one or more additional components, such as the language module, power source 32 and electrode pairs 22 of the integrated component package 10. Alternatively, the integrated component package 10 may include a removable portion to facilitate solely the transfer of the memory/software chipset 34.

While illustrative embodiments of the invention have been illustrated and described, it will be appreciated that various changes can be made therein without departing from the spirit and scope of the invention.

The embodiments of the invention in which an exclusive property or privilege is claimed are defined as follows:

1. An integrated component package, the package comprising:
one or more medical device components operable to be utilized in conjunction with the operation of a medical device; and
at least one transducer device operable to communicate with the medical device.

2. The package as recited in claim 1, wherein the transducer device is an electrotherapy device operable to obtain an electrotherapy signal from the medical device.

3. The package as recited in claim 1, wherein the transducer device is a monitoring device operable to obtain a monitoring signal from a patient.

4. The package as recited in claim 1, wherein the transducer device includes an electrotherapy device operable to obtain an electrotherapy signal from the medical device and a monitoring device operable to obtain a monitoring signal from a patient.

5. The package as recited in claim 4, wherein the transducer device is a combination electrotherapy device and monitoring device.

6. The package as recited in claim 4, wherein the transducer device includes at least one pair of transducers as an electrotherapy device and at least one pair of transducers as a monitoring device.

7. The package as recited in claim 1, wherein the transducer device is a single use electrode set.

8. The package as recited in claim 1, wherein the transducer device is a multiple use electrode set.

9. The package as recited in claim 1, wherein the medical device components include medical supplies.

10. The package as recited in claim 9, wherein the medical supplies are selected from a group consisting of a mouth to mouth barrier, a safety razor, protective gloves, scissors, alcohol swabs, printed instructions, and towels.

11. The package as recited in claim 1, wherein the medical device components include drugs/medications.

12. The package as recited in claim 11, wherein the drugs/medications are selected from a group consisting of oral drugs/medications, injectable drugs/medications, and transdermal drugs/medication.

13. The package as recited in claim 1, wherein the medical device components include devices associated with the operation of the medical device.

14. The package as recited in claim 13, wherein the devices associated with the operation of the medical device are selected from a group consisting of one or more additional transducer devices operable to communicate with the medical device, one or more input devices operable to transfer data to the medical device, an activation device operable to initiate a self-test of the medical device, a user interface control operable to transmit a signal to initiate a delivery of a therapy treatment, a power source operable to deliver power to the medical device, a programmable data source having medical data thereon, a programmable data storage device operable to obtain and store medical device data from the medical device, and a programmable data storage device operable to obtain and store patient data from the medical device.

15. The package as recited in claim 13, wherein the devices associated with the operation of the medical device include a power source operable to deliver power to the medical device, and wherein the integrated component package further includes a power source interface operable to deliver power from the power source to the medical device.

16. The package as recited in claim 15, wherein the power source is a disposable battery.

17. The package as recited in claim 15, wherein the power source is a rechargeable battery.

18. The package as recited in claim 13,
wherein the devices associated with the operation of the medical device include the programmable data source having medical device data thereon, and wherein the integrated component package further includes a data interface operable to transmit the medical device data to the medical device.

19. The package as recited in claim 18, wherein the programmable data source includes software update information operable to update software in the medical device.

20. The package as recited in claim 18, wherein the programmable data source includes identification information for tracking the integrated component package.

21. The package as recited in claim 20, wherein the identification information includes a date of creation or date of expiration.

22. The package as recited in claim 21, wherein the identification information includes a serial number.

23. The package as recited in claim 18, wherein the programmable data source is further operable to obtain and store medical device data from the medical device.

24. The package as recited in claim 13, wherein the one or more medical device components include the user interface control, and wherein the user interface control is a button.

25. The package as recited in claim 13, wherein the one or more medical device components include the input device, and wherein the input device is a microphone.

26. The package as recited in claim 13, wherein the one or more medical device components include the input device, and wherein the input device is a keypad.

27. The package as recited as claim 1, wherein the integrated therapy package is a single use package.

28. The package as recited in claim 1, wherein the medical device is a defibrillator.

29. The package as recited in claim 1, wherein the medical device is a monitoring device.

30. A medical device comprising:
an external medical device component interface operable to connect to a medical device component associated with an integrated component package; and
an external transducer interface operable to be connected to an external transducer device associated with the integrated component package;
wherein the external medical device component interface and the external transducer interface are operable to interface with the integrated component package.

31. The medical device as recited in claim 30, wherein the external medical device component interface is an external power interface operable to receive power from a power source associated with the integrated component package.

32. The medical device as recited in claim 30, wherein the external medical device component interface is an external data interface operable to obtain medical device data from a programmable data source.

33. The medical device as recited in claim 30, wherein the external medical device component interface is selected from a group consisting of an external power interface operable to receive power from a power source associated with the integrated component package, an external data interface operable to obtain medical device data from a programmable data source, an external user interface operable to obtain medical device data from an external data interface, an external user interface operable to obtain patient data from an external data interface, and an external data interface operable to transmit patient and device data to a programmable data source.

34. The medical device as recited in claim 30, wherein the external transducer interface is operable to deliver an electrotherapy signal to the external transducer set.

35. The medical device as recited in claim 30, wherein the external transducer interface is operable to obtain a monitoring signal from the external transducer set.

36. The medical device as recited in claim 30, wherein the medical device is a defibrillator.

37. The medical device as recited in claim 30, wherein the medical device is a monitoring device.

38. The medical device as recited in claim 30, wherein the monitoring device is an ECG monitoring device.

39. A medical device system, the system comprising:
a medical device having an external transducer interface; and
an integrated component package having one or more medical device components operable to be utilized in conjunction with the operation of the medical device, and a transducer device operable to communicate with the external transducer interface of the medical device;
wherein the integrated component package is removably coupled to the medical device.

40. The medical device system as recited in claim 39, wherein the integrated package is a cartridge and wherein the integrated component package engages the medical device.

41. The medical device system as recited in claim 39, wherein the transducer device is an electrotherapy device operable to obtain an electrotherapy signal from the external transducer interface.

42. The medical device system as recited in claim 39, wherein the transducer device is a monitoring device operable to transmit a monitoring signal associated with a patient to the external transducer interface.

43. The medical device system as recited in claim 39, wherein the electrotherapy device is a single use electrode set.

44. The medical device system as recited in claim 39, wherein the medical device components include medical supplies.

45. The medical device system as recited in claim 44, wherein the medical supplies are selected from a group consisting of a mouth to mouth barrier, a safety razor, protective gloves, scissors, alcohol swabs, printed instructions, and towels.

46. The medical device system as recited in claim 39, wherein the medical device components include drugs/medications.

47. The medical device system as recited in claim 46, wherein the drugs/medications are selected from a group consisting of oral drugs/medications, injectable drugs/medications, and transdermal drugs/medication.

48. The medical device system as recited in claim 39, wherein the medical device further includes an external medical device component interface, and wherein the medical device components include devices associated with the operation of the medical device.

49. The medical device system as recited in claim 48, wherein the devices associated with the operation of the medical device are selected from a group consisting of one or more additional transducer devices operable to communicate with the medical device, one or more input devices operable to transfer data to the medical device, an activation device operable to initiate a self-test of the medical device, a user interface control operable to transmit a signal to initiate a delivery of a therapy treatment, a power source operable to deliver power to the medical device, a programmable data source having medical data thereon, a programmable data storage device operable to obtain and store medical device data from the medical device, and a programmable data storage device operable to obtain and store patient data from the medical device.

50. The medical device system as recited in claim 48, wherein the external medical device component interface is an external power interface operable to receive power from a power source associated with the integrated component package.

51. The medical device system as recited in claim 48, wherein the external medical device component interface is an external data interface operable to obtain medical device data from a programmable data source.

52. The medical device system as recited in claim 48, wherein the external medical device component interface is selected from a group consisting of an external power interface operable to receive power from a power source associated with the integrated component package, an external data interface operable to obtain medical device data from a programmable data source, an external a user interface operable to obtain medical device data from the external data interface, and an external data interface operable to transmit patient and device data to a programmable data source.

53. The medical device system as recited in claim 52, wherein the power source is a disposable battery.

54. The medical device system as recited in claim 52, wherein the programmable data source includes software update information operable to update software in the medical device.

55. The medical device system as recited in claim 52, wherein the programmable data source includes identification information for the integrated component package.

56. The medical device system as recited in claim 52, wherein the external data interface is further operable to transmit patient and medical device data to the programmable data source and wherein the programmable data source stores the patient and medical device data.

57. The medical device system as recited in claim 52, wherein the integrated component package is further operable to initiate a status assessment within the medical device.

58. The medical device system as recited in claim 39, wherein the medical device is a defibrillator.

59. The medical device system as recited in claim 39, wherein the medical device is a monitoring system.

60. A medical device system, the system comprising:
a medical device having power interface means and transducer interface means; and
an integrated component package having power source means to deliver a power source to the medical device and transducer means to communicate with the transducer interface means;
wherein the medical device and the integrated component package are removably coupled.

61. The medical device system as recited in claim 60, wherein the medical device further includes data interface means, and where the integrated component package further includes data storage means for delivering medical device data.

62. The medical device system as recited in claim 61, wherein the medical device further includes a second data interface means, and wherein the integrated component package includes a second data storage means to obtain medical device data from the medical device.

63. The medical device system as recited in claim 60, wherein the transducer means is operable to obtain an electrotherapy signal generated by the medical device.

64. The medical device system as recited in claim 60, wherein the transducer means is operable to transmit a monitor signal to the medical devices.

65. The medical device system as recited in claim 60, wherein the integrated component package includes one or more medical device components operable to be utilized in conjunction with the operation of the medical device selected from a group consisting of a mouth to mouth barrier, a safety razor, protective gloves, scissors, alcohol swabs, towels, oral drugs/medications, injectable drugs/medications, transdermal drugs/medication, printed instructions, one or more additional transducer devices operable to communicate with the medical device, one or more input devices operable to transfer data to the medical device, an activation device operable to initiate a self-test of the medical device, and a user interface control operable to transmit a signal to initiate a delivery of a therapy treatment.

66. An integrated component package, the package comprising:
one or more medical device components operable to be utilized in conjunction with the operation of a medical device;
at least one transducer device operable to communicate with the medical device;
a programmable data source having language-specific data thereon, the language-specific data operable to generate a language-specific interface on the medical device; and
a data interface operable to transmit the language-specific information to the medical device.

67. The package as recited in claim 66, wherein the transducer device is a single use electrode set.

68. The package as recited in claim 66, wherein the medical device components include medical supplies.

69. The package as recited in claim 68, wherein the medical supplies are selected from a group consisting of a mouth to mouth barrier, a safety razor, protective gloves, scissors, alcohol swabs, printed instructions, and towels.

70. The package as recited in claim 66, wherein the medical device components include drugs/medications.

71. The package as recited in claim 70, wherein the drugs/medications are selected from a group consisting of oral drugs/medications, injectable drugs/medications, and transdermal drugs/medication.

72. The package as recited in claim 66, wherein the medical device components include devices associated with the operation of the medical device.

73. The package as recited in claim 72, wherein the devices associated with the operation of the medical device are selected from a group consisting of one or more input devices operable to transfer data to the medical device, an activation device operable to initiate a self-test of the medical device, a user interface control operable to transmit a signal to initiate a delivery of a therapy treatment, a power source operable to deliver power to the medical device, a second programmable data source having medical data thereon, a second programmable data storage device operable to obtain and store medical device data from the medical device, and a second programmable data storage device operable to obtain and store medical device data from the medical device.

74. The package as recited in claim 73, wherein the power source is a disposable battery.

75. The package as recited in claim 73, wherein the one or more medical device components include the power source, and wherein the integrated component package further includes a power source interface operable to deliver power from the power source to the medical device.

76. The package as recited in claim 75, wherein the one or more medical device components include the programmable data source having medical device data thereon, and wherein the integrated component package further includes a data interface operable to transmit the medical device data to the medical device.

77. The package as recited in claim 76, wherein the power source interface and the data interface are the same.

78. The package as recited in claim 66, wherein the transducer device is operable to obtain an electrotherapy signal from the medical devices.

79. The package as recited in claim 78, wherein the medical device is a defibrillator.

80. The package as recited in claim 66, wherein the transducer device is operable to transmit a monitoring signal corresponding to a patient.

81. A medical device comprising:
an external transducer interface operable to communicate with a transducer set; and
an external data interface operable to obtain language-specific medical device data from a programmable data source, the language-specific medical device data for providing a language-specific user interface;
wherein the the external transducer interface and the external data interface are operable to interface with one or more external component packages.

82. The medical device as recited in claim 81, further comprising an external medical device component interface, wherein the external medical device component interface is an external power interface operable to receive power from a power source associated with the integrated component package.

83. The medical device as recited in claim 81, further comprising an external medical device component interface, wherein the external medical device component interface is an external data interface operable to obtain medical device data from a programmable data source.

84. The medical device as recited in claim 81 further comprising an external medical device component interface, wherein the external medical device component interface is selected from a group consisting of an external power interface operable to receive power from a power source associated with the integrated component package, an external a user interface operable to obtain the medical device data from the external data interface, an external data interface operable to transmit patient data to a programmable data source, and an external data interface operable to transmit medical device data to a programmable data server.

85. The medical device as recited in claim 81, wherein the external transducer interface is operable to deliver an electrotherapy signal to the external transducer set.

86. The medical device as recited in claim 81, wherein the external transducer interface is operable to obtain a monitoring signal from the external transducer set.

87. The medical device as recited in claim 81, wherein the medical device is a defibrillator.

88. The medical device as recited in claim 81, wherein the medical device is a monitoring device.

89. The medical device as recited in claim 88, wherein the monitoring device is an ECG monitoring device.

90. A medical device system, the system comprising:
a medical device having an external data interface; and
an integrated component package having one or more medical device components operable to be utilized in conjunction with the use of the medical device, a transducer device operable to communicate with the external transducer interface of the medical device, and a programmable data source having language-specific data operable to generate a language-specific user interface on the medical device, the programmable data source operable to transmit the language-specific data to the external data interface of the medical device;
wherein the integrated component package is removably coupled to the medical device.

91. The medical device system as recited in claim 90, wherein the medical device components include medical supplies.

92. The medical device system as recited in claim 91, wherein the medical supplies are selected from a group consisting of a mouth to mouth barrier, a safety razor, protective gloves, scissors, alcohol swabs, printed instructions, and towels.

93. The medical device system as recited in claim 90, wherein the medical device components include drugs/medications.

94. The medical device system as recited in claim 93, wherein the drugs/medications are selected from a group consisting of oral drugs/medications, injectable drugs/medications, and transdermal drugs/medication.

95. The medical device system as recited in claim 90, wherein the medical device further includes an external medical device component interface, and wherein the medical device components include devices associated with the operation of the medical device.

96. The medical device system as recited in claim 95, wherein the devices associated with the operation of the medical device are selected from a group consisting of one or more input devices operable to transfer data to the medical device, an activation device operable to initiate a self-test of the medical device, a user interface control operable to transmit a signal to initiate a delivery of a therapy treatment, a power source operable to deliver power to the medical device, a second programmable data source having medical data thereon, and a second programmable data storage device operable to obtain and store medical device data from the medical device.

97. The medical device system as recited in claim 96, wherein the external medical device component interface is an external power interface operable to receive power from a power source associated with the integrated component package.

98. The medical device system as recited in claim 96, wherein the external medical device component interface is an external data interface operable to obtain medical device data from a programmable data source.

99. The medical device system as recited in claim 96, wherein the external medical device component interface is selected from a group consisting of an external power interface operable to receive power from a power source associated with the integrated component package, an external a user interface operable to obtain the medical device data from the external data interface, and an external data interface operable to transmit patient and device data to a programmable data source.

100. The medical device system as recited in claim 99, wherein the power source is a disposable battery.

101. The medical device system as recited in claim 99, wherein the second programmable data source includes software update information operable to update software in the medical device.

102. The medical device system as recited in claim 99, wherein the second programmable data source includes identification information for the integrated component package.

103. The medical device system as recited in claim 90, wherein the medical device is a defibrillator.

104. The medical device system as recited in claim 90, wherein the medical device is a monitoring device.

105. The medical device system as recited in claim 104, wherein the monitoring device is an ECG monitoring device.
